(19) United States
(12) Patent Application Publication
(10) Pub. No.: US 2025/0108087 A1
(43) Pub. Date: Apr. 3, 2025

(54) VETERINARY PHARMACEUTICAL COMPOSITION

(71) Applicant: BIOLAB SANUS FARMACÊUTICA LTDA, Taboao da Serra (BR)

(72) Inventors: Felipe Moreira da Silva, Guarulhos (BR); Rafael da Fonseca Prietsch, Bragança Paulista (BR)

(73) Assignee: BIOLAB SANUS FARMACEUTICA LTDA, Taboao da Serra, SP (BR)

(21) Appl. No.: 18/832,807

(22) PCT Filed: Dec. 16, 2022
(86) PCT No.: PCT/BR2022/050505

§ 371 Date: Jul. 24, 2024

(30) Foreign Application Priority Data
Jan. 26, 2022 (BR) BR102022001420-5

(51) Int. Cl. A61K 36/84; A61K 9/00; A61K 9/20; A61K 31/405; A61K 36/185

(52) U.S. Cl. CPC A61K 36/84; A61K 9/0056; A61K 9/2009; A61K 9/2013; A61K 9/2018; A61K 9/2031; A61K 9/2054; A61K 9/2068; A61K 31/405; A61K 36/185

(57) ABSTRACT

The present invention relates to: a veterinary pharmaceutical composition, in the form of a palatable tablet, containing a combination of tryptophan, valerian extract and passionflower extract; to the use of the composition for the treatment of noise phobias in companion animals and/or domestic animals, preferably in dogs, more specifically, as well as in rescued wild animals and zoo animals: and to the veterinary pharmaceutical composition in which the pharmaceutically and/or veterinarily acceptable excipients act synergistically to provide a tablet with adequate hardness and low friability.

### FIELD OF THE INVENTION

The present invention relates to a veterinary pharmaceutical composition, in the form of a palatable tablet, containing the combination of tryptophan, valerian extract and passionflower extract; the use of said composition for the treatment of noise phobias in animals, such as companion animals and/or domestic animals, preferably in dogs, as well as a treatment method comprising the administration of said composition to an animal who has noise phobias.

### BACKGROUND OF THE INVENTION

Noise phobias by disturbing stimuli, such as aversive, popping sounds, produced by fireworks and storms with thunder and lightning, are among the most known disturbances associated with panic or phobia responses in companion animals and/or domestic animals, preferably dogs.

Storms with thunder and lightning, fireworks, firearms, shouting, car ignition, among others, often induce undesirable clinical symptoms in animals, especially companion and/or domestic animals, particularly dogs. Said clinical symptoms include excessive salivating, defecating, urinating, destruction, fleeing, shaking, etc. Companion and/or domestic animals include dogs, cats, hamsters, guinea pigs or any other common pet, but preferably dogs, as well as rescued wild animals and zoo animals.

Today, there are various measures and/or therapies to try to control the fear of the companion animals and/or domestic animals in relation to aversive sounds. Among these measures, standing out most are isolated environmental management measures (such as, for example, closing windows and curtains, use of muffling background music, use of specific site as safe haven to which the animal is previously conditioned) to more robust measures, comprising behavioral change and psychoactive agents, both allopathic and homeopathic, nutraceuticals, aromatherapy, synthetic pheromones and even Bach Flower Remedies.

With the purpose of seeking an alternative for treating the noise phobia through disturbing stimuli, such as aversive sounds, the researchers of the present invention developed a veterinary pharmaceutical composition, in the form of a palatable tablet, containing the combination of a nutraceutical (tryptophan) and two herbal medicines (valerian extract and passionflower extract) to be applied in companion animals and/or domestic animals, preferably for dogs, with noise phobias by disturbing stimuli.

However, during the development of the product, in the form of a palatable tablet, containing a combination of a nutraceutical (tryptophan) and two herbal medicines (valerian extract and passionflower extract) major hurdles were identified in obtaining a tablet with adequate hardness and low friability, which was related to the moisture of the active ingredients.

Consequently, it is the object of this invention to provide an adequate composition for the formation of a palatable tablet, which acts synergistically relative to the increase in rigidity (hardness) and decrease of the friability of the tablets, containing a binder and an adsorbent, in the ratio of 1:2, respectively and, accordingly, solve the technical problem identified by the researchers.

### SUMMARY OF THE INVENTION

The aim of the present invention is to provide a veterinary pharmaceutical composition, preferably, but not just in the form of a palatable tablet, containing a combination of a nutraceutical (tryptophan) and two herbal medicines (valerian extract and passionflower extract), especially developed to assist in the prevention and/or treatment of noise phobias by disturbing stimuli in companion animals and/or domestic animals, preferably for dogs. Companion and/or domestic animals include dogs, cats, hamsters, guinea pigs or any other common pet, but preferably dogs, as well as rescued wild animals and zoo animals. In a preferred embodiment, the present invention provides a veterinary pharmaceutical composition, in the form of a palatable tablet, comprising the combination of a nutraceutical (tryptophan) and two herbal medicines (valerian extract and passionflower extract); with pharmaceutically acceptable excipients that act synergistically to provide a tablet with adequate hardness and low friability.

### DETAILED DESCRIPTION OF THE INVENTION

The objective of the present invention is to produce an adequate veterinary pharmaceutical composition for the treatment of phobias in animals.

Said composition is provided preferably in the form of a palatable tablet, with improved hardness and friability, comprising the combination of tryptophan, valerian extract and passionflower extract, to assist in the treatment of noise phobias in animals, preferably companion animals and/or domestic animals, preferably dogs.

The Valerian extract is obtained from valerian, which is the genus of perennial herbaceous plants of the valerianaceae family. Valerian is understood to be any species of this genus, such as, for example, common valerian, or simply valerian, Valerian officinalis, with fragrant inflorescences and thick roots with characteristic and strong odor, from which, adequately treated (macerated, crushed, dried and packaged), herbal medicines having an anxiolytic and calming effect are prepared, classically used in medicine. The genus valerian has various known species, including: Valerian officinalis, Valerian edulis, Valerian acutiloba, Valerian californica, Valerian occidentalis, and others.

The Passionflower extract is obtained from passionflower which is a botanical genus belonging to the Passifloraceae family. The leaves and roots of the passion fruit tree contain maracujina, passiflorina and calmofilase; pharmaceutical principles widely used as sedative ingredients, anti-spasmodic, anti-inflammatory and purifying ingredients. The genus Passiflora has diverse known species, including: Passiflora incarnata, Passiflora edulis, Passiflora alata, Passiflora allardii, Passiflora emarginata, Passiflora margaritae, Passiflora santiagana, and others.

Due to its role in the biosynthesis of serotonin and melatonin, Tryptophan or L-tryptophan, plays an important function in the biochemical processes of sleep and mood, to the extent that it is used as a supplement for treating depression, for example.

In one embodiment, the present invention refers to an adequate veterinary pharmaceutical composition containing the combination of one nutraceutical and two herbal medicines. More specifically comprising:
(a) the phytotherapic plant extract of the genus Valerian,
(b) the phytotherapic plant extract of the genus Passiflora,
(c) the nutraceutical tryptophan or L-tryptophan, and
(d) pharmaceutically and/or veterinarily acceptable excipients.

The Valerian extract, present in the composition, can be selected from any type belonging to the genus Valerian, and further, it may be selected from the group of alcoholic dry extract, hydroalcoholic dry extract or glycolic dry extract. Preferably, the extract of the genus Valerian is in the form of hydroalcoholic dry extract, obtained from the rhizome and root of the plant, of the species Valerian officinalis. The Valerian extract may be present in the composition in the range of 12.5 mg to 100.00 mg, representing about 3.25% to 4.45% (w/w) based on the total weight of the final composition, preferably, in a concentration of 3.65% to 4.05% (w/w) based on the total weight of the final composition of the present invention.

The Passionflower extract, present in the composition, may be selected from any species belonging to the genus Passiflora, and further, it may be selected from the group of alcoholic dry extract, hydroalcoholic dry extract or glycolic dry extract. Preferably, the extract of the genus Passiflora is in the form of hydroalcoholic dry extract, obtained from the aerial parts of the plant, of the species Passiflora incarnata L. and/or Passiflora edulis. The passionflower extract may be present in the composition in the range of 50.00 mg to 400.00 mg, representing about 13.05% to 17.70% (w/w) based on the total weight of the final composition. Preferably, the passionflower extract is present in a concentration of 14.65% to 16.20% (w/w) based on the total weight of the final composition of the present invention.

Tryptophan or L-tryptophan is an essential amino acid that increases the production of serotonin in the central nervous system. Serotonin is an important neurotransmitter that regulates mood, appetite and sleep, and is often used to treat cases of depression or anxiety. Tryptophan or L-tryptophan may be present in the range of 62.50 mg to 500.0 mg, representing about 16.30% to 22.10% (w/w) based on the total weight of the final composition of the present invention. Preferably, tryptophan or L-tryptophan is present in a concentration of 18.30% to 20.20% (w/w) based on the total weight of the final composition of the present invention.

The pharmaceutically and/or veterinarily acceptable excipients comprised in the veterinary pharmaceutical composition of the present invention include, but are not limited to the group selected from: diluents, binders, adsorbents, lubricants, flavorings, enhancers, and mixtures thereof.

For a clearer understanding of the present invention, the following examples are merely illustrative and not meant to limit the scope or underlying principles of the invention in any way.

Examples of diluents include but are not limited to the group selected from anhydrous lactose, monohydrate lactose, spray-dried lactose, microcrystalline cellulose, mannitol, sorbitol, corn starch, sucrose, and mixtures thereof. Preferably, the diluent is a spray-dried lactose, microcrystalline cellulose, mannitol, and mixtures thereof, and may be present in the range of 30.6% to 41.40% (w/w) based on the total weight of the final composition of the present invention. More preferably, present in the range of 34.15% to 37.90% (w/w) based on the total weight of the final composition of the present invention.

Examples of binders include, but are not limited to the group selected from hydroxypropylcellulose, hydroxyethylcellulose, hydroxypropylmethylcellulose, ethylcellulose and povidone. Preferably, the binder is a hydroxypropylcellulose, and may be present in the range of 1.25% to 5.75% (w/w) based on the total weight of the final composition of the present invention. More preferably, present in the range of 4.75% to 5.25% (w/w) based on the total weight of the final composition of the present invention.

Examples of adsorbents include but are not limited to the group selected from aluminum and magnesium metasilicate, bentonite, magnesium carbonate, calcium carbonate, dibasic calcium phosphate dihydrate, tricalcium phosphate and calcium silicate. Preferably, the adsorbent is the aluminum and magnesium metasilicate (Neusilin®) and may be present in the range of 8.50% to 11.50% (w/w) based on the total weight of the final composition of the present invention. More preferably, present in the range of 9.50% to 10.50% (w/w) based on the total weight of the final composition of the present invention.

Examples of lubricants include but are not limited to the group selected from silicon dioxide, talc, polyethylenoglycol (PEG or Macrogol) 8000, magnesium stearate, magnesium stearate vegetal, mineral oil, sodium stearyl fumarate, calcium stearate, stearic acid, zinc stearate, and mixtures thereof. Preferably, the lubricant is silicon dioxide, talc, polyethyleneglycol (PEG or Macrogol) 8000, vegetable magnesium stearate, and mixture thereof, and may be present in the range of 4.25% to 5.75% (w/w) based on the total weight of the final composition of the present invention. More preferably, it is present in the range of 4.75% to 5.25% (w/w) based on the total weight of the final composition of the present invention.

Examples of flavorings include, but are not limited to the group selected from vanilla flavoring, bacon flavoring, meat flavoring, chicken flavoring, lamb flavoring, salmon flavoring, rib flavoring, fish flavoring, and mixtures thereof. Preferably, the flavoring is the vanilla flavoring, bacon flavoring, and mixtures thereof, and may be present in the range of 1.70% to 2.30% (w/w) based on the total weight of the final composition of the present invention. More preferably, present in the range of 1.90% to 2.10% (w/w) based on the total weight of the final composition of the present invention.

Examples of enhancers include, but are not limited to the group selected from pork liver hydrolysate (Palasurance®), pork hydrolysate (D-Tech® 8P), yeast extract (Prosaf®), and mixtures thereof. Preferably, the enhancer is the pork liver hydrolysate (Palasurance®) and may be present in the range of 2.95% to 4.0% (w/w) based on the total weight of the final composition of the present invention. More preferably, present in the range of 3.30% to 3.70% (w/w) based on the total weight of the final composition of the present invention.

During the development of the product containing the combination of tryptophan, valerian extract and passionflower extract, in the form of a palatable tablet, obstacles were identified in obtaining a tablet with adequate hardness and low friability, due to the hygroscopicity of the active ingredients.

In one embodiment of the present invention, the veterinary pharmaceutical composition, in the form of a palatable tablet, comprises:
(a) a plant extract of the genus Valerian,
(b) a plant extract of the genus Passiflora,
(c) a tryptophan or L-tryptophan,
(d) at least one diluent,
(e) a binder,
(f) an adsorbent,
(g) at least one lubricant,
(h) at least one flavoring, and
(i) at least one enhancer.

In a more preferred embodiment of the present invention, the veterinary pharmaceutical composition, in the form of a palatable tablet, comprises:
(a) from 3.25% to 4.45% (w/w) of a plant extract of the genus Valerian,
(b) from 13.05% to 17.70% (w/w) of a plant extract of the genus Passiflora,
(c) from 16.30% to 22.10% (w/w) of a tryptophan or L-tryptophan,
(d) from 30.60% to 41.40% (w/w) of at least one diluent,
(e) from 1.25% to 5.75% (w/w) of a binder,
(f) from 8.50% to 11.50% (w/w) of an adsorbent,
(g) from 4.25% to 5.75% (w/w) of at least one lubricant,
(h) from 1.70% to 2.30% (w/w) of at least one flavoring, and,
(i) from 2.95% to 4.0% (w/w) of at least one enhancer; based on the total weight of the final composition.

In an even more preferred embodiment of the present invention, the veterinary pharmaceutical composition, in the form of a palatable tablet, comprises:
(a) from 3.65% to 4.05% (w/w) of a plant extract of the genus Valerian,
(b) from 14.65% to 16.20% (w/w) of a plant extract of the genus Passiflora,
(c) from 18.30% to 20.20% (w/w) of a tryptophan or L-tryptophan,
(d) from 34.15% to 37.90% (w/w) of at least one diluent,
(e) from 4.75% to 5.25% (w/w) of a binder,
(f) from 9.50% to 10.50% (w/w) of an adsorbent,
(g) from 4.75% to 5.25% (w/w) of at least one lubricant,
(h) from 1.90% to 2.10% (w/w) of at least one flavoring, and,
(i) from 3.30% to 3.70% (w/w) of at least one enhancer;
based on the total weight of the final composition.

During the development of the present invention, the researchers noted that the combination of a binder and an adsorbent, in the ratio 1:2, respectively, surprisingly displayed synergistic activity in obtaining palatable tablets, containing the combination of tryptophan, valerian extract and passionflower extract, with adequate hardness and low friability of the palatable tablets.

The veterinary pharmaceutical composition, in the form of a palatable tablet, containing the combination of tryptophan, valerian extract and passionflower extract are to assist in the treatment of noise phobias in companion animals and/or domestic animals, preferably in dogs. Noise phobias through disturbing stimuli occur due to the sounds produced by fire works, stores with thunder and lightning, firearms, shouting, car ignition, and others.

The present invention also refers to the use of the veterinary pharmaceutical composition, in the form of a palatable tablet, containing the combination of tryptophan, valerian extract and passionflower extract, prepared for assisting in the treatment of noise phobias in animals. Said animals are preferably companion and/or domestic animals, preferably in dogs. Alternatively, a method for treating or relieving noise phobias in animals is also claimed, comprising the administration of a composition described in this patent application to an animal suffering from noise phobias, such as a companion animal and/or domestic animal, preferably in dogs.

The veterinary pharmaceutical compositions of the present invention can be prepared by applying techniques known in the state of the art, as described in the examples. However, a process of obtaining a pharmaceutical composition as described above and in the examples is also an embodiment of the present invention. As can be seen ahead, said process may comprise diverse variations according to the application desired for the end product, however it comprises the essential steps of:
Preparation of a first mixture of the active ingredients, optionally jointly with flavorings and enhancers;
Preparation of a second mixture adding diluents, binding agent and adsorbent agent to the first mixture;
Preparation of a final mixture adding lubricants to the second mixture; and
Compression of the final mixture.

Examples of embodiments of the invention are provided below. Nevertheless, it must be understood that said examples and embodiments are provided with a solely illustrative purpose and that various modifications and/or changes, in light of the embodiments disclosed herein, will be suggestive to a person skilled in the art and should be encompassed within the spirit and scope of this specification and scope of the accompanying claims.

### EXAMPLES

In the examples set out below, various experiments were made with different types of formulations, with the aim of obtaining a veterinary pharmaceutical composition, in the form of a palatable tablet with adequate hardness and low friability, containing the combination of tryptophan, valerian extract and passionflower extract.

### Example 1: Formulations without Binder and without Adsorbent

In a first attempt at formulation, 3 formulations containing the combination of tryptophan, valerian extract and passionflower extract were prepared, and common excipients used to obtain tablets, but without the use of binding agents or adsorbents.

TABLE 1
formulations A, B and C

| | | Formula A | | Formula B | | Formula C | |
|---|---|---|---|---|---|---|---|
| | | QT | Perc. | QT | Perc. | QT | Perc. |
| Components | FUNCTION | (mg) | (%) | (mg) | (%) | (mg) | (%) |
| DRY EXT. | Active | 25.000 | 5.000 | 12.500 | 3.846 | 75.000 | 3.846 |
| VALERIAN | | | | | | | |
| HYDROALCOHOL | | | | | | | |
| DRY EXT. | Active | 100.000 | 20.000 | 50.000 | 15.385 | 300.000 | 15.385 |
| PASSIONFLOWER | | | | | | | |
| HYDROALCOHOL | | | | | | | |
| L-TRYPTOPHAN | Active | 125.000 | 25.000 | 62.500 | 19.231 | 375.000 | 19.231 |
| Vanilla Flavoring | Flavoring | — | — | — | — | 19.500 | 1.000 |
| Bacon Flavoring | Flavoring | 2.750 | 0.550 | 3.250 | 1.000 | — | — |
| Meat Flavoring | Flavoring | — | — | 3.250 | 1.000 | 19.500 | 1.000 |
| Pork liver | Enhancer | — | — | 11.375 | 3.500 | — | — |
| hydrolyzate | | | | | | | |
| (Palasurance ®) | | | | | | | |
| Pork hydrolysate | Enhancer | 33.000 | 6.600 | — | — | 70.200 | 3.600 |
| (D-Tech ® 8P) | | | | | | | |
| Spray dried lactose | Diluent | 27.790 | 5.558 | 24.625 | 7.577 | 102.375 | 5.250 |
| Microcrystalline | Diluent | 62.300 | 12.460 | 62.500 | 19.231 | 501.900 | 25.738 |
| cellulose PH102 | | | | | | | |
| Mannitol | Diluent | 113.160 | 22.632 | 48.750 | 15.000 | — | — |
| Sorbitol | Diluent | — | — | 30.000 | 9.231 | 384.150 | 19.700 |
| Silicon dioxide | Lubricant | 5.500 | 1.100 | 3.250 | 1.000 | 20.475 | 1.050 |
| Talc | Lubricant | — | — | 3.250 | 1.000 | 20.475 | 1.050 |
| Polyethyleneglycol | Lubricant | — | — | 6.500 | 2.000 | 40.950 | 2.100 |
| 8000 (PEG 8000) | | | | | | | |
| Vegetable | Lubricant | 5.500 | 1.100 | 3.250 | 1.000 | 20.475 | 1.050 |
| Magnesium | | | | | | | |
| Stearate | | | | | | | |

| | | | | | | |
|---|---|---|---|---|---|---|
| TOTAL | 500.000 | 100.000 | 325.000 | 100.000 | 1.950.000 | 100.000 |

### Manufacturing Process:

The active ingredients, flavorings and enhancers are sieved through mesh 20, and mixed for 10 minutes (mixture 1). The diluents are sieved through mesh 20, and added to the prior mixture (mixture 1), and mixed for 10 minutes in a V-mixer (mixture 2). The lubricants (silicon dioxide, talc and PEG 8000, if present) are sieved through mesh 20, and added to the prior mixture (mixture 2) and mixed for 10 minutes in a V-mixer (mixture 3). The lubricant magnesium stearate is sieved through mesh 40, added to the prior mixture (mixture 3), and mixed in a V-mixer for 3 minutes (mixture 4). Next, the mixture obtained (mixture 4) is subject to compression in a rotary compressor.

Each of the formulations A, B and C, were tested after the compression process. However, it was only possible to carry out the moisture test, since there was no adequate formation of the tablets, with capping thereof occurring. The results of these analyses are indicated in the Table of tests described in example 5.

### Example 2: Formulations with Binding Agent

In a second attempt at formulation, 3 formulations containing the combination of tryptophan, valerian extract and passionflower extract were prepared, with the addition of a binding agent in the composition.

TABLE 2
Formulations D, E and F

| | | Formula D | | Formula E | | Formula F | |
|---|---|---|---|---|---|---|---|
| | | QT | Perc. | QT | Perc. | QT | Perc. |
| Components | FUNCTION | (mg) | (%) | (mg) | (%) | (mg) | (%) |
| DRY EXT. | Active | 25.000 | 3.846 | 12.500 | 3.846 | 75.000 | 3.846 |
| VALERIAN | | | | | | | |
| HYDROALCOHOL | | | | | | | |
| DRY EXT. | Active | 100.000 | 15.385 | 50.000 | 15.385 | 300.000 | 15.385 |
| PASSIONFLOWER | | | | | | | |
| HYDROALCOHOL | | | | | | | |
| L-TRYPTOPHAN | Active | 125.000 | 19.231 | 62.500 | 19.231 | 375.000 | 19.231 |
| Vanilla Flavoring | Flavoring | 6.500 | 1.000 | — | — | 19.500 | 1.000 |
| Bacon Flavoring | Flavoring | 6.500 | 1.000 | 3.250 | 1.000 | 19.500 | 1.000 |
| Meat Flavoring | Flavoring | — | — | 3.250 | 1.000 | — | — |
| Pork liver | Enhancer | — | — | 11.700 | 3.600 | — | — |
| hydrolyzate | | | | | | | |
| (Palasurance ®) | | | | | | | |
| Pork hydrolysate | Enhancer | 23.300 | 3.585 | — | — | 78.000 | 4.000 |
| (D-Tech ® 8P) | | | | | | | |
| Spray dried | Diluent | 30.875 | 4.750 | 31.000 | 9.538 | 101.400 | 5.200 |
| lactose | | | | | | | |
| Microcrystalline | Diluent | 145.325 | 22.358 | 70.800 | 21.785 | 380.950 | 19.536 |
| cellulose PH102 | | | | | | | |
| Mannitol | Diluent | — | — | 47.500 | 14.615 | — | — |
| Sorbitol | Diluent | 123.500 | 19.000 | — | — | 409.500 | 21.000 |
| Hydroxypropyl- | Binder | 32.500 | 5.000 | — | — | — | — |
| cellulose (HPC) | | | | | | | |
| Hydroxyethyl- | Binder | — | — | 16.250 | 5.000 | — | — |
| cellulose | | | | | | | |
| Hydroxypropyl- | Binder | — | — | — | — | 97.500 | 5.000 |
| methylcellulose | | | | | | | |
| Silicon dioxide | Lubricant | 6.500 | 1.000 | 3.250 | 1.000 | 17.550 | 0.900 |
| Talc | Lubricant | 6.500 | 1.000 | 3.250 | 1.000 | 17.550 | 0.900 |
| Polyethyleneglycol | Lubricant | 12.000 | 1.846 | 6.500 | 2.000 | 41.000 | 2.103 |
| 8000 (PEG 8000) | | | | | | | |
| Vegetable | Lubricant | 6.500 | 1.000 | 3.250 | 1.000 | 17.550 | 0.900 |
| Magnesium | | | | | | | |
| Stearate | | | | | | | |

| | | | | | | |
|---|---|---|---|---|---|---|
| TOTAL | 650.000 | 100.000 | 325.000 | 100.000 | 1.950.000 | 100.000 |

### Manufacturing Process:

The active ingredients, flavorings and enhancers are sieved through mesh 20, and mixed for 10 minutes (mixture 1). The diluents and the binding agent are sieved through mesh 20 and added to the prior mixture (mixture 1) and mixed for 10 minutes in a V-mixer (mixture 2). The lubricants (silicon dioxide, talc and PEG 8000, if present) are sieved through mesh 20, and added to the prior mixture (mixture 2) and mixed for 10 minutes in a V-mixer (mixture 3). The lubricant magnesium stearate is sieved through mesh 40, added to the prior mixture (mixture 3), and mixed in a V-mixer for 3 minutes (mixture 4). Next, the mixture obtained (mixture 4) is subject to compression in a rotary compressor.

Each of the formulations D, E and F were tested after the compression process. However, it was only possible to carry out the moisture test, since there was no adequate formation of the tablets, with “capping” thereof occurring. The results of these analyses are indicated in the Table of tests described in example 5.

### Example 3: Formulations with Adsorbent Agent

In a third attempt at formulation, 3 formulations containing the combination of tryptophan, valerian extract and passionflower extract were prepared, with the addition of adsorbent agent in the composition.

TABLE 3
Formulations G, H and I

| | | Formula G | | Formula H | | Formula I | |
|---|---|---|---|---|---|---|---|
| | | QT | Perc. | QT | Perc. | QT | Perc. |
| Components | FUNCTION | (mg) | (%) | (mg) | (%) | (mg) | (%) |
| DRY EXT. | Active | 25.000 | 4.167 | 12.500 | 3.846 | 75.000 | 3.846 |
| VALERIAN | | | | | | | |
| HYDROALCOHOL | | | | | | | |
| DRY EXT. | Active | 100.000 | 16.667 | 50.000 | 15.385 | 300.000 | 15.385 |
| PASSIFLORA | | | | | | | |
| HYDROALCOHOL | | | | | | | |
| L-TRYPTOPHAN | Active | 125.000 | 20.833 | 62.500 | 19.231 | 375.000 | 19.231 |
| Vanilla Flavoring | Flavoring | — | — | — | — | 19.500 | 1.000 |
| Bacon Flavoring | Flavoring | 6.500 | 1.083 | 3.250 | 1.000 | — | — |
| Meat Flavoring | Flavoring | — | — | 3.250 | 1.000 | 19.500 | 1.000 |
| Pork liver | Enhancer | 39.000 | 6.500 | 11.375 | 3.500 | — | — |
| hydrolyzate | | | | | | | |
| (Palasurance ®) | | | | | | | |
| Pork hydrolysate | Enhancer | — | — | — | — | 70.200 | 3.600 |
| (D-Tech ® 8P) | | | | | | | |
| Spray dried | Diluent | 51.400 | 8.567 | 24.625 | 7.577 | 167.050 | 8.567 |
| lactose | | | | | | | |
| Microcrystalline | Diluent | 77.800 | 12.967 | 63.375 | 19.500 | 315.625 | 16.186 |
| cellulose PH102 | | | | | | | |
| Mannitol | Diluent | 77.800 | 12.967 | 28.750 | 8.846 | 315.625 | 16.186 |
| Sorbitol | Diluent | — | — | 15.000 | 4.615 | — | — |
| Aluminum and | Adsorbent | 65.000 | 10.833 | — | — | — | — |
| Magnesium | | | | | | | |
| metasilicate | | | | | | | |
| (Neusilin ®) | | | | | | | |
| Bentonite | Adsorbent | — | — | 34.125 | 10.500 | — | — |
| Magnesium | Adsorbent | — | — | — | — | 195.000 | 10.000 |
| carbonate | | | | | | | |
| Silicon dioxide | Lubricant | 6.500 | 1.083 | 3.250 | 1.000 | 19.500 | 1.000 |
| Talc | Lubricant | 6.500 | 1.083 | 3.250 | 1.000 | 19.500 | 1.000 |
| Polyethylene- | Lubricant | 13.000 | 2.167 | 6.500 | 2.000 | 39.000 | 2.000 |
| glycol 8000 | | | | | | | |
| (PEG 8000) | | | | | | | |
| Vegetable | Lubricant | 6.500 | 1.083 | 3.250 | 1.000 | 19.500 | 1.000 |
| Magnesium | | | | | | | |
| Stearate | | | | | | | |

| | | | | | | |
|---|---|---|---|---|---|---|
| TOTAL | 600.000 | 100.000 | 325.000 | 100.000 | 1.950.000 | 100.000 |

### Manufacturing Process:

The active ingredients, flavorings and enhancers are sieved through mesh 20, and mixed for 10 minutes (mixture 1). The diluents and the adsorbent agent are sieved through mesh 20, and added to the prior mixture (mixture 1), and mixed for 10 minutes in a V-mixer (mixture 2). The lubricants (silicon dioxide, talc and PEG 8000, if present) are sieved through mesh 20, and added to the prior mixture (mixture 2) and mixed for 10 minutes in a V-mixer (mixture 3). The lubricant magnesium stearate is sieved through mesh 40, added to the prior mixture (mixture 3), and mixed in a V-mixer for 3 minutes (mixture 4). Next, the mixture obtained (mixture 4) is subject to compression in a rotary compressor.

Each of the formulations G, H and I, were tested after the compression process. However, it was only possible to carry out the moisture test, since there was no adequate formation of the tablets, with “capping” thereof occurring. The results of these analyses are indicated in the Table of tests described in example 5.

### Example 4: Formulations with Binding Agent and Adsorbent

In a fourth attempt at formulation, 6 formulations containing the combination of tryptophan, valerian extract and passionflower extract were prepared, with the addition of a binding agent and adsorbent in the composition.

TABLE 4
Formulations J, K, L, M, N and O

| | | Formula J | | Formula K | | Formula L | |
|---|---|---|---|---|---|---|---|
| | | QT | Perc. | QT | Perc. | QT | Perc. |
| Components | FUNCTION | (mg) | (%) | (mg) | (%) | (mg) | (%) |
| DRY EXT. | Active | 25.000 | 3.846 | 25.000 | 3.846 | 25.000 | 3.846 |
| VALERIAN | | | | | | | |
| HYDROALCOHOL | | | | | | | |
| DRY EXT. | Active | 100.000 | 15.385 | 100.000 | 15.385 | 100.000 | 15.385 |
| PASSIONFLOWER | | | | | | | |
| HYDROALCOHOL | | | | | | | |
| L-TRYPTOPHAN | Active | 125.000 | 19.231 | 125.000 | 19.231 | 125.000 | 19.231 |
| Vanilla Flavoring | Flavoring | 6.500 | 1.000 | 6.500 | 1.000 | 6.500 | 1.000 |
| Bacon Flavoring | Flavoring | 6.500 | 1.000 | 6.500 | 1.000 | 6.500 | 1.000 |
| Pork liver | Enhancer | 22.750 | 3.500 | 22.750 | 3.500 | 22.750 | 3.500 |
| hydrolyzate | | | | | | | |
| (Palasurance ®) | | | | | | | |
| Spray dried | Diluent | 32.500 | 5.000 | 32.500 | 5.000 | 32.500 | 5.000 |
| lactose | | | | | | | |
| Microcrystalline | Diluent | 80.000 | 12.308 | 80.000 | 12.308 | 80.000 | 12.308 |
| cellulose PH102 | | | | | | | |
| Mannitol | Diluent | 121.750 | 18.731 | 121.750 | 18.731 | 121.750 | 18.731 |
| Aluminum and | Adsorbent | — | — | — | — | 65.000 | 10.000 |
| Magnesium | | | | | | | |
| Metasilicate | | | | | | | |
| (Neusilin ®) | | | | | | | |
| Bentonite | Adsorbent | 65.000 | 10.000 | 65.000 | 10.000 | — | — |
| Hydroxypropyl- | Binder | 32.500 | 5.000 | — | — | — | — |
| cellulose (HPC) | | | | | | | |
| Hydroxypropyl- | Binder | — | — | 32.500 | 5.000 | 32.500 | 5.000 |
| methylcellulose | | | | | | | |
| Silicon dioxide | Lubricant | 6.500 | 1.000 | 6.500 | 1.000 | 6.500 | 1.000 |
| Talc | Lubricant | 6.500 | 1.000 | 6.500 | 1.000 | 6.500 | 1.000 |
| Polyethylene- | Lubricant | 13.000 | 2.000 | 13.000 | 2.000 | 13.000 | 2.000 |
| glycol 8000 | | | | | | | |
| (PEG 8000) | | | | | | | |
| Vegetable | Lubricant | 6.500 | 1.000 | 6.500 | 1.000 | 6.500 | 1.000 |
| Magnesium | | | | | | | |
| Stearate | | | | | | | |

| TOTAL | | 650.000 | 100.000 | 650.000 | 100.000 | 650.000 | 100.000 |
|---|---|---|---|---|---|---|---|
| | | Formula M | | Formula N | | Formula O | |
| | | QT | Perc. | QT | Perc. | QT | Perc. |
| | Components | (mg) | (%) | (mg) | (%) | (mg) | (%) |
| | DRY EXT. | 25.000 | 3.846 | 25.000 | 3.846 | 25.000 | 3.846 |
| | VALERIAN | | | | | | |
| | HYDROALCOHOL | | | | | | |
| | DRY EXT. | 100.000 | 15.385 | 100.000 | 15.385 | 100.000 | 15.385 |
| | PASSIONFLOWER | | | | | | |
| | HYDROALCOHOL | | | | | | |
| | L-TRYPTOPHAN | 125.000 | 19.231 | 125.000 | 19.231 | 125.000 | 19.231 |
| | Vanilla Flavoring | 6.500 | 1.000 | 6.500 | 1.000 | 6.500 | 1.000 |
| | Bacon Flavoring | 6.500 | 1.000 | 6.500 | 1.000 | 6.500 | 1.000 |
| | Pork liver | 22.750 | 3.500 | 22.750 | 3.500 | 22.750 | 3.500 |
| | hydrolyzate | | | | | | |
| | (Palasurance ®) | | | | | | |
| | Spray dried | 32.500 | 5.000 | 32.500 | 5.000 | 32.500 | 5.000 |
| | lactose | | | | | | |
| | Microcrystalline | 80.000 | 12.308 | 80.000 | 12.308 | 80.000 | 12.308 |
| | cellulose PH102 | | | | | | |
| | Mannitol | 89.250 | 13.731 | 121.750 | 18.731 | 89.250 | 13.731 |
| | Aluminum and | 65.000 | 10.000 | 65.000 | 10.000 | 97.500 | 15.000 |
| | Magnesium | | | | | | |
| | Metasilicate | | | | | | |
| | (Neusilin ®) | | | | | | |
| | Bentonite | — | — | — | — | — | — |
| | Hydroxypropyl- | 65.000 | 10.000 | 32.500 | 5.000 | 32.500 | 5.000 |
| | cellulose (HPC) | | | | | | |
| | Hydroxypropyl- | — | — | — | — | — | — |
| | methylcellulose | | | | | | |
| | Silicon dioxide | 6.500 | 1.000 | 6.500 | 1.000 | 6.500 | 1.000 |
| | Talc | 6.500 | 1.000 | 6.500 | 1.000 | 6.500 | 1.000 |
| | Polyethylene- | 13.000 | 2.000 | 13.000 | 2.000 | 13.000 | 2.000 |
| | glycol 8000 | | | | | | |
| | (PEG 8000) | | | | | | |
| | Vegetable | 6.500 | 1.000 | 6.500 | 1.000 | 6.500 | 1.000 |
| | Magnesium | | | | | | |
| | Stearate | | | | | | |
| | TOTAL | 650.000 | 100.000 | 650.000 | 100.000 | 650.000 | 100.000 |

### Manufacturing Process:

Valerian, Passionflower and L-tryptophan, flavorings and enhancers are sieved through mesh 20, and mixed for 10 minutes (mixture 1). The diluents, the binding agent and the adsorbent agent are sieved through mesh 20, and added to the prior mixture (mixture 1), and mixed for 10 minutes in a V-mixer (mixture 2). The lubricants (silicon dioxide, talc and PEG 8000, if present) are sieved through mesh 20, and added to the prior mixture (mixture 2) and mixed for 10 minutes in a V-mixer (mixture 3). The lubricant magnesium stearate is sieved through mesh 40, added to the prior mixture (mixture 3), and mixed in a V-mixer for 3 minutes (mixture 4). Next, the mixture obtained (mixture 4) is subject to compression in a rotary compressor.

Each of the formulations J, K, L, M, N and O, were tested after the compression process. The results of these analyses are indicated in the Table of tests described in example 5.

### Example 5: Result of the Quality Tests of the Composition

The tests carried out to check the quality of the composition were: hardness, friability, disintegration and moisture (loss through drying). The methodology adopted in performing the quality tests followed the American Pharmacopoeia (USP 39)

TABLE 5
Result of the Tests

| | | Specifi- | Form. | Form. | Form. | Form. | Form. | Form. | Form. |
|---|---|---|---|---|---|---|---|---|---|
| Tests | Ref. | cation | A | B | C | D | E | F. | G. |
| Hardness | USP 39 | 80N to | NA | NA | NA | NA | NA | NA | NA |
| | | 115N | | | | | | | |
| Friability | USP 39 | Max. 1.5% | NA | NA | NA | NA | NA | NA | NA |
| Disinte- | USP 39 | Max. 30 | NA | NA | NA | NA | NA | NA | NA |
| gration in | | minutes | | | | | | | |
| water at | | | | | | | | | |
| 37° C. | | | | | | | | | |
| Moisture | USP 39 | ≤1.80% | 4.68% | 4.27% | 4.39% | 4.12% | 4.45% | 4.32% | 2.31% |
| (Loss | | | | | | | | | |
| through | | | | | | | | | |
| drying) | | | | | | | | | |

| | Form. | Form. | Form. | Form. | Form. | Form. | Form. | Form. |
|---|---|---|---|---|---|---|---|---|
| Tests | H | I | J | K | L | M | N | O |
| Hardness | NA | NA | 83N | 86N | 85N | 84N | 90N | 82N |
| Friability | NA | NA | 1.80% | 1.40% | 1.23% | 1.3% | 0.88% | 1.65% |
| Disinte- | NA | NA | 41 min. | 43 min. | 29 min. | 16 min. | 19 min | 23 min. |
| gration in | | | | | | | | |
| water at | | | | | | | | |
| 37° C. | | | | | | | | |
| Moisture | 2.65% | 2.93% | 2.41% | 2.53% | 1.77% | 1.97% | 1.67% | 1.52% |
| (Loss | | | | | | | | |
| through | | | | | | | | |
| drying) | | | | | | | | |

Legend: NA (Not applicable): Test not carried out, as it was not possible to obtain the tablet.

Therefore, based on the data obtained during development of the composition, object of the present invention, including the results of the tests on hardness, friability, disintegration and moisture (loss through drying) tests performed, the researchers noted that the absence of binder and adsorbent, or the use of just one binder or adsorbent, did not solve the technical problem identified, since there was no adequate formation of the tablets, with “capping” thereof occurring. However, with the combined use of a binding agent and an adsorbent agent, it was possible to obtain the tablets in a satisfactory manner.

Surprisingly, the researchers noted that by combining one binding agent with an adsorbent agent, in the optimal ratio of 1:2, respectively, it was possible to obtain palatable tablets, containing the combination of tryptophan, valerian extract and passionflower extract, within the quality specifications of the tests for hardness, friability, disintegration time and moisture (see Table 5).

Unless defined otherwise, all the technical and scientific terms used in the present document shall have the same meaning as commonly understood by a person skilled in the art to which the present invention belongs. Furthermore, although the invention has been described with reference to some embodiments thereof, it shall be understood that alterations and similar modifications can be made, but are within the state of the art.

1. A veterinary pharmaceutical composition, in the form of a palatable tablet, characterized in that it comprises:
(a) a plant extract of the genus Valerian,
(b) a plant extract of the genus Passiflora,
(c) a tryptophan or L-tryptophan, and
(d) pharmaceutically and/or veterinarily acceptable excipients.

2. The veterinary pharmaceutical composition according to claim 1, characterized in that the plant extract of the genus Valerian is an alcoholic dry extract, hydroalcoholic dry extract and/or glycolic dry extract.

3. The veterinary pharmaceutical composition according to claim 2, characterized in that the plant extract of the genus Valerian is selected from the group of the species Valeriana officinalis, Valeriana edulis, Valeriana acutiloba, Valeriana californica, and Valeriana occidentalis.

4. The veterinary pharmaceutical composition according to claim 1, characterized in that the plant extract of the genus Passiflora is an alcoholic dry extract, hydroalcoholic dry extract and/or glycolic dry extract.

5. The veterinary pharmaceutical composition according to claim 4, characterized in that the plant extract of the genus Passiflora is selected from the group of the species Passiflora incarnata, Passiflora edulis, Passiflora alata, Passiflora allardii, Passiflora emarginata, Passiflora margaritae, and Passiflora santiagana.

6. The veterinary pharmaceutical composition according to claim 1, characterized in that the pharmaceutically and/or veterinarily acceptable excipients are selected from the group consisting of diluents, binders, adsorbents, lubricants, flavorings, enhancers, and mixtures thereof.

7. The veterinary pharmaceutical composition according to claim 1, characterized in that it comprises:
(a) a plant extract of the genus Valerian,
(b) a plant extract of the genus Passiflora,
(c) a tryptophan or L-tryptophan,
(d) at least one diluent,
(e) a binder,
(f) an adsorbent,
(g) at least one lubricant,
(h) at least one flavoring, and
(i) at least one enhancer.

8. The veterinary pharmaceutical composition according to claim 7, characterized in that it comprises:
(a) from 3.25% to 4.45% (w/w) of a plant extract of the genus Valerian,
(b) from 13.05% to 17.70% (w/w) of a plant extract of the genus Passiflora,
(c) from 16.30% to 22.10% (w/w) of a tryptophan or L-tryptophan,
(d) from 30.60% to 41.40% (w/w) of at least one diluent,
(e) from 1.25% to 5.75% (w/w) of a binder,
(f) from 80.50% to 11.50% (w/w) of an adsorbent,
(g) from 4.25% to 5.75% (w/w) of at least one lubricant,
(h) from 1.70% to 2.30% (w/w) of at least one flavoring, and,
(i) from 2.95% to 4.0% (w/w) of at least one enhancer;
based on the total weight of the final composition.

9. The veterinary pharmaceutical composition according to claim 8, characterized in that it comprises:
(a) from 3.65% to 4.05% (w/w) of a plant extract of the genus Valerian,
(b) from 14.65% to 16.20% (w/w) of a plant extract of the genus Passiflora,
(c) from 18.30% to 20.20% (w/w) of a tryptophan or L-tryptophan,
(d) from 34.15% to 37.90% (w/w) of at least one diluent,
(e) from 4.75% to 5.25% (w/w) of a binder,
(f) from 9.50% to 10.50% (w/w) of an adsorbent,
(g) from 4.75% to 5.25% (w/w) of at least one lubricant,
(h) from 1.90% to 2.10% (w/w) of at least one flavoring, and,
(i) from 3.30% to 3.70% (w/w) of at least one enhancer;
based on the total weight of the final composition.

10. The veterinary pharmaceutical composition according to claim 7, characterized in that the binder and adsorbent are in the ratio of 1:2.

11. The veterinary pharmaceutical composition according to claim 7, characterized in that the diluent is selected from the group consisting of: anhydrous lactose, monohydrate lactose, spray-dried lactose, microcrystalline cellulose, mannitol, sorbitol, corn starch, sucrose, and mixtures thereof, preferably wherein the diluent is spray-dried lactose, microcrystalline cellulose, mannitol, or mixtures thereof.

12. (canceled)

13. The veterinary pharmaceutical composition according to claim 7, characterized in that the binder is selected from the group consisting of: hydroxypropylcellulose, hydroxyethylcellulose, hydroxypropylmethylcellulose, ethylcellulose, and povidone, preferably wherein the binder is hydroxypropylcellulose.

14. (canceled)

15. The veterinary pharmaceutical composition according to claim 7, characterized in that the adsorbent is selected from the group consisting of: aluminum and magnesium metasilicate, bentonite, magnesium carbonate, calcium carbonate, dibasic calcium phosphate dihydrate, tricalcium phosphate and calcium silicate, preferably wherein the adsorbent is the aluminum and magnesium metasilicate.

16. (canceled)

17. The veterinary pharmaceutical composition according to claim 7, characterized in that the lubricant is selected from the group consisting of: silicon dioxide, talc, polyethylenoglycol (PEG or Macrogol) 8000, magnesium stearate, vegetable magnesium stearate, mineral oil, sodium stearyl fumarate, calcium stearate, stearic acid, zinc stearate, and mixtures thereof, preferably wherein the lubricant is silicon dioxide, talc, polethyleneglycol (PEG or Macrogol) 8000, vegetable magnesium stearate, or mixtures thereof.

18. (canceled)

19. The veterinary pharmaceutical composition according to claim 7, characterized in that the flavoring is selected from the group consisting of: vanilla flavoring, bacon flavoring, meat flavoring, chicken flavoring, lamb flavoring, salmon flavoring, rib flavoring, fish flavoring, and mixtures thereof, preferably wherein the flavoring is vanilla flavoring, bacon flavoring, or mixtures thereof.

20. (canceled)

21. The veterinary pharmaceutical composition according to claim 7, characterized in that the enhancer is selected from the group consisting of: pork liver hydrolysate (Palasurance®), pork hydrolysate (D Tech® 8P), yeast extract (Prosaf®), and mixtures thereof, preferably wherein the enhancer is the pork liver hydrolysate (Palasurance®).

22-26. (canceled)

27. A method for treating or relieving noise phobias in animals comprising administering a composition as defined in claim 1 to an animal suffering from noise phobias.

28. A process of obtaining a pharmaceutical composition as defined in claim 1 comprising:
Preparation of a first mixture of the active ingredients, optionally jointly with flavorings and enhancers;
Preparation of a second mixture adding diluents, binding agent and adsorbent agent to the first mixture; and
Preparation of a final mixture adding lubricants to the second mixture; and
Compression of the final mixture.
